(19) United States
(12) Patent Application Publication
(10) Pub. No.: US 2026/0306588 A1
(43) Pub. Date: Oct. 8, 2026

(54) COMPOSITIONS AND USES OF GLP-1-GIP RECEPTOR AGONISTS

(71) Applicant: Maison Blaise Labs, LLC, Las Vegas, NV (US)

(72) Inventor: Gabrielle Rios, Las Vegas, NV (US)

(21) Appl. No.: 19/637,850

(22) Filed: Apr. 2, 2026

Related U.S. Application Data
(60) Provisional application No. 63/783,912, filed on Apr. 4, 2025.

(51) Int. Cl. A61K 38/26; A61K 31/27; A61P 1/16

(52) U.S. Cl. CPC A61K 38/26; A61K 31/27; A61P 1/16

(57) ABSTRACT

Described herein are methods, pharmaceutical compositions, and kits with GLP-1-GIP receptor agonists for ameliorating at least one symptom or underlying conditions associated with post-cholecystectomy syndrome (PCS). The methods, pharmaceutical compositions, and kits are also useful in pain management and/or treatment by modulating pain perception via vagal nerve and nucleus tractus solitarius pathways and to reduce opioid dependency in patients with pain.

### CROSS-REFERENCE TO RELATED APPLICATIONS

This application claims the benefit of priority of U.S. Provisional Patent Application No. 63/783,912, filed Apr. 4, 2025, which is hereby expressly incorporated by reference in its entirety.

### BACKGROUND

### Field

The present disclosure is related to compositions and uses of GLP-1-GIP receptor agonists.

### Description of the Related Art

Post-cholecystectomy syndrome (PCS) is a complex condition that includes a range of biliary and non-biliary causes.

### SUMMARY

Some embodiments provided herein relate to methods for preventing, ameliorating, or treating post-cholecystectomy syndrome (PCS). In some embodiments, the methods include identifying a patient in need thereof and administering to the patient a therapeutically effective dose of a GLP-1-GIP receptor agonist. In some embodiments, the methods prevent, ameliorate, or treat at least one symptom or underlying condition associated with PCS.

In some embodiments, the at least one symptom or underlying condition associated with PCS is one or more biliary or non-biliary complications associated with PCS. In some embodiments, the one or more biliary complications include bile duct injury, bile duct stricture, Sphincter of Oddi Dysfunction (SOD), bile acid malabsorption, cystic duct remnant syndrome, or retained common bile duct stones. In some embodiments, the one or more non-biliary complications include irritable bowel syndrome, gastroesophageal reflux disease, peptic ulcer disease, chronic pancreatitis, intercostal neuritis, functional GI disorders, psychiatric/neurological disorders, wound neuroma, coronary artery disease, mesenteric ischemia, diverticulitis, dumping syndrome, pancreatic cancer, and bile duct cancer.

In some embodiments, the GLP-1-GIP receptor agonist improves bile acid metabolism and gut motility, thereby alleviating diarrhea, bloating, and food intolerance associated with PCS.

In some embodiments, the methods include reducing sphincter spasms, biliary obstruction, and hepatobiliary congestion. In some embodiments, the methods include managing post-cholecystectomy biliary dysfunction and bile duct motility disorders. In some embodiments, the methods include reducing pancreatic ductal hypertension and/or preventing secondary pancreatitis by optimizing biliary drainage.

In some embodiments, the methods further include reducing CNS-level pain. In some embodiments, the GLP-1-GIP receptor agonist modulates pain signaling through the vagus nerve and central nervous system, providing relief of neurogenic and functional pain following PCS. In some embodiments, the methods include modulating pain perception via vagal nerve pathways to reduce opioid dependency in patients with biliary pain. In some embodiments, the GLP-1-GIP receptor agonist activates receptors in the nucleus tractus solitarius (NTS) and other brainstem structures, thereby attenuating central sensitization and modulating pain via the gut-brain axis. In some embodiments, the methods include modulating pain perception via NTS to reduce opioid dependency in patients with biliary pain. In some embodiments, the GLP-1-GIP receptor agonist exerts an effect through vagus nerve modulation, anti-inflammatory mechanisms, smooth muscle relaxation, improved bile flow and metabolism, and/or normalization of liver enzymes. In some embodiments, the dual GLP-1/GIP receptor agonist provides therapeutic benefit through one or more of vagal nerve modulation of pain perception via the gut-brain axis, reduction of neuroinflammation or visceral hypersensitivity, smooth muscle relaxation of the biliary or intestinal sphincters, enhancement of bile flow and acid metabolism, and/or stabilization of hepatobiliary enzyme levels.

In some embodiments, the methods include maintaining long-term stability of liver enzymes, liver volume reduction, and/or liver fibrosis reduction. In some embodiments, the methods include reducing elevated levels of liver enzymes that are indicators of liver function by about 75-fold to about 100-fold. In some embodiments, the methods include reducing elevated levels of liver enzymes that are indicators of liver function to within normal ranges. In some embodiments, administration of the GLP-1-GIP receptor agonist results in normalization of ALT/AST levels, resolution of abdominal pain, avoidance of ERCP or surgery, and/or reduction in opioid use.

In some embodiments, the therapeutically effective dose ranges from about 1 mg/day to about 5 mg/day. In some embodiments, the therapeutically effective dose is 2.5 mg/day. In some embodiments, the therapeutically effective dose is a dose that reduces elevated levels of liver enzymes that are indicators of liver function to within normal ranges. In some embodiments, the GLP-1-GIP receptor agonist is tirzepatide.

In some embodiments, the methods include delivering the GLP-1-GIP receptor agonist via sublingual film, intranasal spray, or both.

In some embodiments, the methods further include co-administering methocarbamol to enhance neuromuscular relaxation and reduce spasmodic biliary pain. In some embodiments, the methods further include administering methocarbamol to treat smooth muscle-related or spasmodic abdominal pain.

Some embodiments provided herein relate to pharmaceutical compositions. In some embodiments, the pharmaceutical compositions are used for preventing, ameliorating, or treating a post-cholecystectomy syndrome (PCS). In some embodiments, the pharmaceutical compositions include a dose of a GLP-1-GIP receptor agonist and a pharmaceutically acceptable carrier. In some embodiments, the at least one symptom or underlying condition associated with PCS is one or more biliary or non-biliary complications associated with PCS.

In some embodiments, the one or more biliary complications include bile duct injury, bile duct stricture, Sphincter of Oddi dysfunction, bile acid malabsorption, cystic duct remnant syndrome, or retained common bile duct stones. In some embodiments, the one or more non-biliary complications include irritable bowel syndrome, gastroesophageal reflux disease, peptic ulcer disease, chronic pancreatitis, intercostal neuritis, functional GI disorders, psychiatric/neurological disorders, wound neuroma, coronary artery disease, mesenteric ischemia, diverticulitis, dumping syndrome, pancreatic cancer, and/or bile duct cancer.

In some embodiments, the dose is therapeutically effective in improving bile acid metabolism and gut motility, thereby alleviating diarrhea, bloating, and/or food intolerance associated with PCS.

In some embodiments, the dose is therapeutically effective in reducing sphincter spasms, biliary obstruction, and/or hepatobiliary congestion. In some embodiments, the dose is therapeutically effective in post-cholecystectomy biliary dysfunction and/or bile duct motility disorders. In some embodiments, the dose is therapeutically effective in reducing pancreatic ductal hypertension and/or preventing secondary pancreatitis by optimizing biliary drainage.

In some embodiments, the dose is therapeutically effective in reducing CNS-level pain. In some embodiments, the dose is therapeutically effective in modulating pain signaling through the vagus nerve and central nervous system, providing relief of neurogenic and functional pain following PCS. In some embodiments, the dose is therapeutically effective in modulating pain perception via vagal nerve pathways to reduce opioid dependency in patients with biliary pain. In some embodiments, the dose is therapeutically effective in activating receptors in the nucleus tractus solitarius (NTS) and other brainstem structures, thereby attenuating central sensitization and modulating pain via the gut-brain axis. In some embodiments, the dose is therapeutically effective in modulating pain perception via NTS to reduce opioid dependency in patients with biliary pain. In some embodiments, the dose is therapeutically effective in exerting an effect through vagus nerve modulation, anti-inflammatory mechanisms, smooth muscle relaxation, improved bile flow and metabolism, and/or normalization of liver enzymes. In some embodiments, the dose is therapeutically effective in providing therapeutic benefit through one or more of vagal nerve modulation of pain perception via the gut-brain axis, reduction of neuroinflammation or visceral hypersensitivity, smooth muscle relaxation of the biliary or intestinal sphincters, enhancement of bile flow and acid metabolism, and/or stabilization of hepatobiliary enzyme levels.

In some embodiments, the dose is therapeutically effective in long-term stabilization of liver enzymes, liver volume reduction, and/or liver fibrosis reduction. In some embodiments, the dose is therapeutically effective in reducing elevated levels of liver enzymes that are indicators of liver function by about 75-fold to about 100-fold. In some embodiments, the dose is therapeutically effective in reducing elevated levels of liver enzymes that are indicators of liver function to within normal ranges. In some embodiments, the dose is therapeutically effective in normalization of ALT/AST levels, resolution of abdominal pain, avoidance of ERCP or surgery, and/or reduction in opioid use.

In some embodiments, the dose ranges from about 1 mg/day to about 15 mg/day. In some embodiments, the dose is 2.5 mg/day. In some embodiments, the dose is a therapeutically effective dose. In some embodiments, the GLP-1-GIP receptor agonist is tirzepatide.

In some embodiments, the pharmaceutical composition is formulated for delivery via a sublingual film, intranasal spray, or both.

In some embodiments, the pharmaceutical composition further includes methocarbamol to enhance neuromuscular relaxation and reduce spasmodic biliary pain. In some embodiments, the pharmaceutical composition further includes methocarbamol to treat smooth muscle-related or spasmodic abdominal pain.

Some embodiments provided herein relate to kits. In some embodiments, the kits prevent, ameliorate, or treat a post-cholecystectomy syndrome (PCS). In some embodiments, the kits include a pharmaceutical composition as described herein and instructions for administering the pharmaceutical composition. In some embodiments, the at least one symptom or underlying condition associated with PCS is one or more biliary or non-biliary complications associated with PCS. In some embodiments, the one or more biliary complications include bile duct injury, bile duct stricture, Sphincter of Oddi dysfunction, bile acid malabsorption, cystic duct remnant syndrome, and/or retained common bile duct stones. In some embodiments, the one or more non-biliary complications include irritable bowel syndrome, gastroesophageal reflux disease, peptic ulcer disease, chronic pancreatitis, intercostal neuritis, functional GI disorders, psychiatric/neurological disorders, wound neuroma, coronary artery disease, mesenteric ischemia, diverticulitis, dumping syndrome, pancreatic cancer, and/or bile duct cancer. In some embodiments, the pharmaceutical composition is formulated as an oral solution, sublingual film, intranasal spray, or intravenous formulation. In some embodiments, the kit further includes methocarbamol formulated for co-administration with the GLP-1-GIP receptor agonist. In some embodiments, the pharmaceutical composition is formulated as an oral solution, sublingual film, intranasal spray, or intravenous formulation. In some embodiments, the kit further includes methocarbamol formulated for co-administration with the GLP-1-GIP receptor agonist.

Some embodiments provided herein related to methods for identifying an individual for GLP-1/GIP-based pain therapy. In some embodiments, the methods include assessing a history of cholecystectomy, presence of a pain, exclusion of structural abnormalities in the individual, and/or measuring bile acid levels or vagal tone indicators in the individual. In some embodiments, the pain is chronic abdominal pain. In some embodiments, an intensity of the pain is assessed using a visual analogue scale (VAS). In some embodiments, the methods further include treating the individual with a pharmaceutical composition comprising a GLP-1-GIP receptor agonist.

Some embodiments provided herein relate to methods for enhancing bile duct drainage and/or neuroimmune modulation. In some embodiments, the methods include identifying a patient with a post-cholecystectomy syndrome (PCS) and administering to the patient a therapeutically effective dose of a GLP-1-GIP receptor agonist. In some embodiments, the GLP-1-GIP receptor agonist produces a synergistic effect greater than a sum of an effect produced by GLP-1 receptor agonist and an effect produced by GIP receptor agonist.

Some embodiments provided herein relate to methods for modulating gut-brain axis for pain management. In some embodiments, the methods include identifying a patient in need thereof and administering to the patient a therapeutically effective dose of a GLP-1-GIP receptor agonist. In some embodiments, the GLP-1-GIP receptor agonist activates the GLP-1 receptor in the nucleus tractus solitarius (NTS), thereby dampening central sensitization and reducing neurogenic visceral pain.

Some embodiments provided herein relate to methods for treating chronic abdominal or visceral pain in a subject in need thereof. In some embodiments, the methods include administering a therapeutically effective amount of a dual GLP-1/GIP receptor agonist, thereby reducing pain severity regardless of biliary, gastrointestinal, neurologic, and/or post-surgical origin.

Some embodiments provided herein relate to methods for reducing or treating a pain in a subject. In some embodiments, the methods include administering to a subject a therapeutically effective amount of a dual GLP-1/GIP receptor agonist, thereby reducing or treating the pain. In some embodiments, the pain is neuropathic, functional gastrointestinal, and/or neurovisceral in origin. In some embodiments, the pain arises from PCS, intercostal neuritis, wound neuroma, and/or central sensitization. In some embodiments, the subject reports decreased pain severity as measured by a validated scale within 4-12 weeks of treatment. In some embodiments, the pain is modulated by activation of GLP-1 receptors on vagal afferent neurons and central structures including the nucleus tractus solitarius (NTS), thereby interrupting gut-brain pain signaling and decreasing nociceptive transmission. In some embodiments, the treatment results in a reduction in opioid usage by at least 50% within three months of initiation, complete opioid discontinuation in at least 6 months, or avoidance of narcotic use altogether. In some embodiments, the reduction in opioid usage by at least 50% within three months of initiation, complete opioid discontinuation in at least 6 months, or avoidance of narcotic use altogether, is maintained for about 1, 2, 3, 4, 5, 6, 7, 8, 9, 10, 11, 12, 13, 14, 15, 16, 17, 18, 19, 20, 21, 22, 23, or 24 months, or 1, 2, 3, 4, 5, 6, 7, 8, 9, 10 or more years, or for a period of time that is within a range defined by any two of the aforementioned values. For example, in some embodiments, the reduction in opioid usage by at least 50% within three months of initiation, complete opioid discontinuation in at least 6 months, or avoidance of narcotic use altogether, is maintained for between about 1-24 months, 1-18, 1-12, 1-6, 1-3, 1-2, 2-24, 2-18, 2-12, 2-6, 2-3, 3-24, 3-18, 3-12, 3-6, 6-24, 6-18, 6-12, 12-24, 12-18, or 18-24 months.

Some embodiments provided herein relate to methods for reducing opioid usage in a patient suffering from post-cholecystectomy pain. In some embodiments, the methods include administering a dual GLP-1/GIP receptor agonist to modulate pain via central vagal pathways and reduce nociceptive signaling.

Some embodiments herein relate to methods of managing chronic abdominal or visceral pain without opioids. In some embodiments, the methods include administering a dual GLP-1/GIP receptor agonist. In some embodiments, the subject's opioid dosage is reduced by at least 50% within three months of initiation. In some embodiments, the reduction in opioid usage by at least 50% within three months of initiation is maintained for about 1, 2, 3, 4, 5, 6, 7, 8, 9, 10, 11, 12, 13, 14, 15, 16, 17, 18, 19, 20, 21, 22, 23, or 24 months, or 1, 2, 3, 4, 5, 6, 7, 8, 9, 10 or more years, or for a period of time that is within a range defined by any two of the aforementioned values. For example, in some embodiments, the reduction in opioid usage by at least 50% within three months of initiation is maintained for between about 1-24 months, 1-18, 1-12, 1-6, 1-3, 1-2, 2-24, 2-18, 2-12, 2-6, 2-3, 3-24, 3-18, 3-12, 3-6, 6-24, 6-18, 6-12, 12-24, 12-18, or 18-24 months.

Some embodiments provided herein relate to methods of treating chronic abdominal pain in a subject in need thereof. In some embodiments, the methods include administering a dual GLP-1/GIP receptor agonist. In some embodiments, the pain arises from a gastrointestinal, hepatobiliary, neurologic, and/or post-surgical source. In some embodiments, the treatment reduces or eliminates the need for opioid analgesics. In some embodiments, the methods further include administration of methocarbamol to alleviate spasmodic or smooth muscle-related abdominal pain. In some embodiments, the methods enhance the efficacy of the GLP-1/GIP receptor agonist in non-opioid pain relief. In some embodiments, the subject demonstrates or achieves ≥70% reduction in baseline Visual Analog Scale (VAS) pain scores, return to normal daily activity without narcotic support, and absence of further invasive biliary or GI intervention within a 6-month period.

Some embodiments provided herein are related to diagnostic methods. In some embodiments, the methods include identifying subjects for GLP-1/GIP agonist-based pain therapy. In some embodiments, the diagnostic methods include obtaining a surgical history including cholecystectomy or biliary interventions, assessing chronic right upper quadrant or epigastric pain, excluding obstructive pathology via imaging, and optionally measuring vagal tone or bile acid markers to stratify for treatment responses.

### BRIEF DESCRIPTION OF THE DRAWINGS

FIG. 1 is an embodiment of a graph showing plasma levels AST (SGOT) and ALT (SGPT) over time in the patient of Example 1.

FIG. 2 is an embodiment of a graph showing plasma levels AST (SGOT) over time in the patient of Example 1.

FIG. 3 is an embodiment of a graph showing plasma levels ALT (SGPT) over time in the patient of Example 1.

### DETAILED DESCRIPTION

The present disclosure is related to use of GLP-1-GIP receptor agonists for the treatment of Post-Cholecystectomy Syndrome (PCS) and related biliary and gastrointestinal disorders.

Post-cholecystectomy syndrome (PCS) encompasses a wide range of symptoms and conditions arising, for example, after gallbladder removal (cholecystectomy), and includes both biliary and non-biliary etiologies. PCS may manifest immediately following surgery or may arise months or years later, and may be transient, intermittent, or chronic in nature. The syndrome may include persistent, recurrent, or new symptoms that are similar to those experienced prior to cholecystectomy, or may include new gastrointestinal or systemic symptoms that develop following removal of the gallbladder.

PCS may arise due to biliary and non-biliary causes. Biliary causes comprise bile duct injury, bile duct stricture, Sphincter of Oddi Dysfunction (SOD), bile acid malabsorption, cystic duct remnant syndrome, or retained common bile duct stones. Non-biliary causes comprise irritable bowel syndrome, gastroesophageal reflux disease, peptic ulcer disease, chronic pancreatitis, intercostal neuritis, functional GI disorders, psychiatric/neurological disorders, wound neuroma, coronary artery disease, mesenteric ischemia, diverticulitis, dumping syndrome, pancreatic cancer, and bile duct cancer. In some cases, altered biliary flow dynamics resulting from the absence of the gallbladder reservoir function may lead to continuous bile flow into the intestine, which may contribute to diarrhea, bile acid malabsorption, mucosal irritation, altered gut motility, and changes in gut microbiota composition. Chronic exposure of the upper gastrointestinal tract to bile may also contribute to gastritis, duodenitis, or gastroesophageal reflux symptoms.

Non-biliary etiologies of PCS may include, for example, pancreatitis, pancreatic insufficiency, peptic ulcer disease, gastroesophageal reflux disease (GERD), functional gastrointestinal disorders such as irritable bowel syndrome (IBS), functional dyspepsia, small intestinal bacterial overgrowth (SIBO), bile acid diarrhea, hepatic disease, and adhesions or other post-surgical anatomical or motility-related complications. In some cases, symptoms may be multifactorial and involve a combination of biliary and non-biliary causes.

Symptoms associated with PCS may include abdominal pain (e.g., right upper quadrant pain, epigastric pain), dyspepsia, bloating, flatulence, nausea, vomiting, diarrhea, steatorrhea, constipation, early satiety, food intolerance (particularly to fatty foods), weight loss, fatigue, and malabsorption of fats and fat-soluble vitamins. Some patients may experience bile acid diarrhea characterized by frequent loose stools, urgency, and irritation of the lower gastrointestinal tract. Other patients may experience predominantly upper gastrointestinal symptoms such as reflux, heartburn, or epigastric discomfort.

In addition to gastrointestinal symptoms, PCS may be associated with metabolic and microbiome-related changes. For example, continuous bile flow into the intestine may alter bile acid composition and enterohepatic circulation, which may in turn influence lipid metabolism, glucose metabolism, gut hormone signaling, and microbial populations within the small intestine and colon. Such changes may contribute to systemic effects including metabolic dysregulation, inflammation, and altered nutrient absorption.

Diagnosis of PCS may involve imaging (e.g., ultrasound, MRCP, ERCP), laboratory testing (e.g., liver function tests, pancreatic enzymes), endoscopy, bile acid testing, stool testing, and functional gastrointestinal evaluations. However, in many patients, no single structural abnormality is identified, and PCS may be diagnosed based on symptoms and exclusion of other conditions.

Management of PCS depends on the underlying cause and may include dietary modification (e.g., reduced fat diet), bile acid sequestrants, antispasmodics, proton pump inhibitors, antibiotics (e.g., for SIBO), pancreatic enzyme supplementation, endoscopic procedures, or surgical revision in select cases. In some patients, symptoms persist despite standard therapies, indicating a need for improved diagnostic methods, targeted therapies, microbiome-based interventions, bile acid modulation therapies, and personalized treatment approaches.

Accordingly, there remains a need for improved compositions, methods, and systems for diagnosing, managing, and treating post-cholecystectomy syndrome, including approaches that address bile acid metabolism, gut microbiome modulation, intestinal barrier function, inflammation, motility, and nutrient absorption in patients who have undergone cholecystectomy.

In some embodiments, PCS represents the primary or root clinical condition from which multiple secondary conditions, complications, or differential diagnoses may arise. Such secondary conditions may include, but are not limited to, Sphincter of Oddi Dysfunction (SOD), bile duct strictures, retained or recurrent common bile duct stones, bile acid malabsorption, biliary dyskinesia, chronic pancreatitis, pancreatic insufficiency, small intestinal bacterial overgrowth (SIBO), functional gastrointestinal disorders, and combinations thereof. In this context, SOD and other biliary or pancreatic complications may be considered manifestations, subsets, sequelae, or associated conditions of PCS rather than wholly independent disease states. Accordingly, therapeutic strategies directed toward the underlying physiological and biochemical disturbances associated with PCS may provide treatment benefits across both biliary and non-biliary etiologies, including those conditions traditionally classified as SOD.

While SOD is commonly recognized as a subset or specific manifestation within the broader clinical spectrum of PCS, PCS encompasses a wider range of etiologies and pathophysiological mechanisms. These include, for example, retained or recurrent biliary stones, cystic duct remnant syndrome, bile duct injury, bile duct stricture, bile leakage, papillary stenosis, bile acid malabsorption, altered bile acid composition, disrupted enterohepatic circulation, hepatic steatosis, functional gastrointestinal disorders, motility disorders, microbiome alterations, and inflammatory conditions of the hepatobiliary and gastrointestinal systems. In many patients, PCS may be multifactorial, involving both structural and functional abnormalities, as well as metabolic and microbiome-related changes that arise following removal of the gallbladder and the resulting loss of bile storage and regulated bile release.

SOD is a disorder characterized by abnormal motility, spasm, hypertensive contraction, impaired relaxation, or discoordination of the sphincter of Oddi, which may lead to intermittent or sustained biliary obstruction, pancreatic duct obstruction, biliary-type pain, pancreatitis, elevated liver enzymes including aspartate aminotransferase (AST) and alanine aminotransferase (ALT), ductal dilation, and impaired bile and pancreatic juice flow into the duodenum. SOD may be classified into biliary-type and pancreatic-type disorders, and may involve structural stenosis, functional obstruction, or a combination thereof. In post-cholecystectomy patients, SOD may be more prevalent due to altered biliary pressure dynamics and continuous bile flow, which may contribute to sphincter dysfunction, inflammation, fibrosis, or neuromuscular dysregulation of the sphincter apparatus.

Traditional treatments for SOD include endoscopic retrograde cholangiopancreatography (ERCP) with sphincterotomy, balloon dilation, or stent placement. While such interventions may provide symptom relief in some patients, these procedures are invasive, carry procedural risks (including pancreatitis, perforation, bleeding, and infection), and may not address the underlying motility disorder, inflammatory processes, hormonal signaling abnormalities, or bile acid dysregulation contributing to the condition. Pharmacologic treatments such as calcium channel blockers, nitrates, antispasmodics, and botulinum toxin injections have been used with variable success and may be limited by side effects or short duration of action.

Glucagon has been used in acute and intraoperative settings to relax the Sphincter of Oddi, indicating that hormonal signaling pathways can modulate sphincter tone, biliary pressure, and bile flow. This observation suggests that glucagon signaling pathways, glucagon receptors, and glucagon-like peptide pathways (including GLP-1, GLP-2, and related analogs, agonists, or modulators) may play a role in regulating biliary motility, sphincter relaxation, hepatic bile production, gallbladder-independent bile flow regulation, and pancreaticobiliary coordination. Prior patent literature (e.g., US 2013/0096059) describes the use of GLP-1 receptor agonists for biliary motility disorders; however, there remains a need for improved, non-invasive, and long-term treatment options for patients suffering from SOD, PCS, hepatomegaly, impaired bile flow, bile acid dysregulation, and related hepatobiliary and gastrointestinal disorders.

In some embodiments, treatment of PCS and associated conditions may involve modulation of bile flow, bile acid composition, enterohepatic circulation, sphincter of Oddi tone, hepatic metabolism, pancreatic secretion, gastrointestinal motility, microbiome composition, intestinal barrier function, inflammation, or combinations thereof. By targeting one or more of these underlying physiological systems, treatment may provide symptom relief and disease modification across a broad range of PCS-associated conditions, including both biliary and non-biliary manifestations, rather than treating SOD or other conditions in isolation.

Accordingly, in some embodiments, the present disclosure is directed to methods of treating PCS as a systemic hepatobiliary and gastrointestinal disorder with multiple downstream manifestations, including SOD, bile acid diarrhea, chronic abdominal pain, pancreatitis, dyspepsia, reflux, microbiome dysbiosis, and metabolic disturbances, thereby providing a more comprehensive therapeutic approach than treatments directed solely to sphincterotomy or localized biliary interventions.

In some embodiments, the present disclosure is related to methods, pharmaceutical compositions, and kits comprising GLP-1-GIP receptor agonists that address a significant unmet medical need for chronic post-surgical and gastrointestinal pain management.

In some embodiments, the present disclosure is related to methods, pharmaceutical compositions, and kits comprising GLP-1-GIP receptor agonists that provide multimodal therapeutic effects GLP-1-GIP receptor agonists including, but not limited to vagal neuromodulation, anti-inflammatory activity, smooth muscle relaxation, and bile acid regulatory effects.

In some embodiments, the present disclosure is related to methods, pharmaceutical compositions, and kits comprising GLP-1-GIP receptor agonists for sustained resolution of right upper quadrant pain, normalization of liver enzymes, cessation of procedural/surgical interventions, and avoidance of further procedural/surgical interventions following therapy with GLP-1-GIP receptor agonists. In some exemplary embodiments, the present disclosure is related to sustained resolution of right upper quadrant pain, normalization of liver enzymes, cessation of procedural/surgical interventions, and avoidance of further procedural/surgical interventions following tirzepatide therapy.

In some embodiments, the present disclosure is related to methods, pharmaceutical compositions, and kits comprising GLP-1-GIP receptor agonists that provide broad neuromodulatory, anti-inflammatory, pro-motility, and bile acid regulatory effects of GLP-1-GIP agonists for multiple etiologies under the umbrella of PCS treatment.

In some embodiments, the present disclosure is related to methods, pharmaceutical compositions, and kits comprising GLP-1-GIP receptor agonists that target PCS as the principal clinical indication. In some embodiments, the present disclosure is related to methods, pharmaceutical compositions, and kits comprising tirzepatide to target PCS as the principal clinical indication.

In some embodiments, the present disclosure is related to methods, pharmaceutical compositions, and kits comprising GLP-1-GIP receptor agonists for the treatment of PCS and related pain conditions.

Treatment of pain is currently underserved, particularly in the post-surgical and functional pain spaces including, but not limited to, neurological pain, gastrointestinal pain, and pain associated with and/or leading to mental health conditions. In some embodiments, the present disclosure is related to methods, pharmaceutical compositions, and kits comprising GLP-1-GIP receptor agonists for chronic pain treatment. In some embodiments, the present disclosure is related to methods, pharmaceutical compositions, and kits comprising GLP-1-GIP receptor agonists for non-opioid/opioid chronic pain treatment.

In some exemplary embodiments, the methods, pharmaceutical compositions, and kits comprising GLP-1-GIP receptor agonists like tirzepatide demonstrate broad efficacy via gut-brain axis modulation, smooth muscle relaxation, anti-inflammatory effects, and bile acid regulation, which can provide treatment benefits in a variety of PCS-related mechanisms.

In some embodiments, administration of the compositions described herein results in one or more clinically meaningful improvements in biochemical, physiological, and patient-reported outcomes associated with hepatobiliary and gastrointestinal function. Such improvements may include, for example, a reduction, normalization, or stabilization of liver enzyme levels, including hepatic transaminases such as alanine aminotransferase (ALT) and/or aspartate aminotransferase (AST). In certain embodiments, reductions in ALT and/or AST may reflect decreased hepatocellular injury, reduced biliary pressure, improved bile flow, improved hepatic metabolism, and/or resolution of inflammation associated with biliary obstruction, sphincter dysfunction, or post-cholecystectomy physiological changes.

In some embodiments, administration of the compositions described herein results in a reduction in pain, including but not limited to abdominal pain, right upper quadrant pain, epigastric pain, and/or spasmodic or colicky pain associated with biliary dyskinesia, sphincter of Oddi dysfunction, or altered gastrointestinal motility. Pain reduction may be characterized by decreased frequency, intensity, and/or duration of pain episodes, and may be assessed using standardized pain scales, patient-reported outcomes, or reduced need for rescue medications.

In some embodiments, administration of the compositions described herein results in a decrease in dependence on opioid or opioid-like analgesics. Such decrease may include reduced dosage, reduced frequency of use, delayed initiation, or complete cessation of opioid therapy, thereby reducing the risk of opioid-associated adverse effects, tolerance, dependence, and/or withdrawal.

In some embodiments, administration of the compositions described herein results in improvements in patient-reported quality of life measures, including increased energy levels, reduced fatigue, improved sleep quality, improved sleep duration, and/or improved sleep architecture. Improvements in sleep may be associated with reductions in nocturnal pain, decreased gastrointestinal discomfort, normalization of circadian metabolic signaling, and/or modulation of neurohormonal pathways.

In some embodiments, administration of the compositions described herein results in improved cognitive function and mental clarity, including improvements in concentration, attention, memory, and/or reduction in symptoms commonly described as “brain fog.” Such improvements may be associated with reduced systemic inflammation, improved metabolic homeostasis, improved nutrient absorption, and/or modulation of gut-brain axis signaling.

In some embodiments, administration of the compositions described herein results in an overall improvement in patient well-being, including enhanced mood, reduced anxiety, improved physical functioning, and increased ability to perform daily activities. Such improvements may be measured using validated quality-of-life instruments, functional assessments, or patient-reported outcome measures.

In some embodiments, administration of the compositions described herein results in improvement in bile function. Improvements in bile function may include, for example, increased bile flow, improved bile composition, improved bile acid balance, enhanced enterohepatic circulation, reduced bile stasis, reduced biliary pressure, improved coordination of bile secretion and intestinal delivery, and/or improved emulsification and absorption of dietary lipids and fat-soluble vitamins. In certain embodiments, such improvements may further result in reductions in bile acid malabsorption, diarrhea, steatorrhea, bloating, and/or dyspeptic symptoms.

In some embodiments, one or more of the foregoing effects may occur independently or in combination, and may be observed over acute, subacute, or chronic administration periods. In certain embodiments, such improvements may be dose-dependent, time-dependent, patient-specific, and/or correlated with underlying disease etiology, including biliary, pancreatic, hepatic, metabolic, or functional gastrointestinal components associated with post-cholecystectomy syndrome or related disorders.

Some embodiments herein provide coverage of the full differential diagnosis and treatment spectrum of PCS based on GLP-1-GIP receptor agonist therapies. Some exemplary embodiments herein provide coverage of the full differential diagnosis and treatment spectrum of PCS based on GLP-1-GIP receptor agonist therapies including tirzepatide.

Table 1 is a non-limiting list of benefits provided by GLP-1-GIP receptor agonists in pain management/treatment:

TABLE 1

| Area of applicability | Benefit |
|---|---|
| Pain | Management and/or treatment of functional pain, |
| | neuropathic/neurological pain, post-surgical pain, |
| | visceral pain, mental health-related pain |
| Mechanism of management/treatment of pain | Via gut-brain axis, via vagal tone, via anti- |
| | inflammatory mechanisms |
| Outcome of pain management/treatment | Opioid use reduction, fewer procedures/ |
| | interventions, use of VAS for pain assessment |
| Indication (regardless of etiology) | Chronic pain, post-surgery pain |
| Diagnostics | Algorithm and/or biomarker-based selection of |
| | patients |

The present disclosure is related to a novel use of GLP-1-GIP receptor agonists, such as tirzepatide, for treating PCS, such as SOD and other biliary disorders, including bile duct motility dysfunction, post-cholecystectomy syndrome, and functional biliary obstruction. Unlike standard interventions, GLP-1-GIP receptor agonists provide a non-invasive, long-term solution by modulating sphincter function and improving biliary flow, while also engaging the gut-brain axis via vagal nerve pathways to reduce visceral pain and neuroinflammation. There is no recognition in the art of the superior effect of GLP-1-GIP agonists for SOD relief, liver enlargement reduction, and bile flow optimization and vagal-mediated neuromodulation. The combination of prolonged enzyme stabilization, sustained sphincter relaxation, liver volume reduction, liver fibrosis reduction, visceral pain control, non-invasive administration, and brain-gut modulation and enhanced quality of life differentiates embodiments of the methods and compositions described herein from current treatments.

The present disclosure is related to unexpectedly and significantly superior effects of the GLP-1-GIP receptor agonists for PCS, such as SOD and other biliary disorders, including but not limited to, bile duct motility dysfunction, post-cholecystectomy syndrome, and functional biliary obstruction. The present disclosure is related to unexpectedly and significantly superior effects of GLP-1-GIP receptor agonists for PCS, including SOD relief, liver enlargement reduction, and bile flow optimization. In terms of mechanism of action, the GLP-1-GIP receptor agonists activate GLP-1 and GIP receptors, providing a synergistic mechanism of action, and thus yielding unexpectedly and significantly superior effects for treating PCS, such as SOD and other biliary disorders as compared to either a GLP-1 receptor agonist or a GIP receptor agonist individually.

In contrast to current treatments, the GLP-1-GIP receptor agonists of the present disclosure provide several unexpected therapeutic benefits including, but not limited to, a combination of prolonged enzyme stabilization, sustained sphincter relaxation, liver volume reduction, liver fibrosis reduction, visceral pain control, and non-invasive administration.

In some embodiments, the present disclosure is related to methods, pharmaceutical compositions, and kits comprising GLP-1-GIP receptor agonists for PCS, such as SOD and other biliary disorders, including but not limited to, bile duct motility dysfunction, post-cholecystectomy syndrome, and functional biliary obstruction.

GLP-1 and GIP receptors modulate smooth muscle tone, leading to prolonged relaxation and improved bile drainage and causing sustained relaxation of SOD. In some embodiments, methods, pharmaceutical compositions, and kits comprising GLP-1-GIP receptor agonists modulate smooth muscle tone, leading to prolonged relaxation and improved bile drainage and causing sustained relaxation of the Sphincter of Oddi.

Activation of GLP-1 and GIP receptors cause enhanced bile flow and reduced hepatobiliary congestion by causing increased bile acid secretion and prevention of bile stagnation to reduce hepatobiliary stress and liver enlargement. In some embodiments, methods, pharmaceutical compositions, and kits comprising GLP-1-GIP receptor agonists provide enhanced bile flow and reduced hepatobiliary congestion by causing increased bile acid secretion and prevention of bile stagnation to reduce hepatobiliary stress and liver enlargement.

GLP-1 receptor activation decreases oxidative stress and inflammation, preventing hepatocyte injury and abnormal liver function tests by reduction of inflammation and stabilization of liver enzyme levels (e.g., AST and ALT). In some embodiments, methods, pharmaceutical compositions, and kits comprising GLP-1-GIP receptor agonists decrease oxidative stress and inflammation, preventing hepatocyte injury and abnormal liver function tests by reduction of inflammation and stabilization of liver enzyme levels (e.g., AST and ALT).

By improving bile drainage, GLP-1-GIP therapy may reduce pancreatic ductal hypertension, preventing low-grade pancreatitis associated with SOD thereby preventing secondary pancreatic dysfunction. In some embodiments, methods, pharmaceutical compositions, and kits comprising GLP-1-GIP receptor agonists improve bile drainage to reduce pancreatic ductal hypertension, preventing low-grade pancreatitis associated with SOD thereby preventing secondary pancreatic dysfunction.

GLP-1 receptor activation in the vagus nerve reduces visceral pain, eliminating the need for narcotic pain management in SOD patients resulting in pain modulation via the gut-brain axis. In some embodiments, methods, pharmaceutical compositions, and kits comprising GLP-1-GIP receptor agonists reduce visceral pain, eliminating the need for narcotic pain management in SOD patients resulting in pain modulation in the vagus nerve via the gut-brain axis. Without being limited to any particular theory, this provides a mechanistic basis for elimination of opioid dependency and narcotic use.

GLP-1 receptor activation in the vagus nerve influences central and peripheral pain circuits, modulating visceral sensitivity, and reducing neurogenic inflammation. In some embodiments, methods, pharmaceutical compositions, and kits comprising GLP-1-GIP receptor agonists provide pain modulation via the gut-brain axis. Without being limited to any particular theory, this provides a mechanistic basis for elimination of opioid dependency and narcotic use.

Without being limited to any particular theory, unlike existing treatments that act solely on mechanical or local gastrointestinal targets, the methods, pharmaceutical compositions, and kits comprising GLP-1-GIP receptor agonists engage the bidirectional communication between the enteric nervous system and the brain. GLP-1 receptor agonists cross the blood-brain barrier and act on central nuclei including the nucleus tractus solitarius (NTS), where afferent vagal inputs converge. This enables a broader therapeutic action not only resolving local biliary obstruction but attenuating the central sensitization and chronic pain syndrome associated with PCS.

In some embodiments, the GLP-1-GIP receptor agonists include, but are not limited to, tirzepatide. Other GLP-1-GIP receptor agonists are within the scope of this disclosure.

In some embodiments, the dose for the GLP-1-GIP receptor agonists is defined based on a therapeutically effective dose (TED). In some embodiments, the TED ranges from about 1 mg/day to about 15 mg/day. In some embodiments, the TED ranges between about 1 mg/day and about 2.5 mg/day. In some embodiments, the TED ranges between about 2.5 mg/day and about 5 mg/day. In some embodiments, the TED ranges between about 5 mg/day and about 7.5 mg/day. In some embodiments, the TED ranges between about 7.5 mg/day and about 10 mg/day. In some embodiments, the TED ranges between about 10 mg/day and about 12.5 mg/day. In some embodiments, the TED ranges between about 12.5 mg/day and about 15 mg/day. In some embodiments, the TED is about 1, 1.5, 2, 2.5, 3, 3.5, 4, 4.5, 5, 6, 7, 8, 9, 10, 11, 12, 13, 14, or 15 mg/day, or within a range defined by any two of the aforementioned values.

In some embodiments, the TED ranges from about 0.015 mg/kg of body weight to about 0.225 mg/kg. In some embodiments, the TED ranges between about 0.015 mg/kg and about 0.0375 mg/kg. In some embodiments, the TED ranges between about 0.0375 mg/kg and about 0.075 mg/kg. In some embodiments, the TED ranges between about 0.1 mg/kg and about 0.15 mg/kg. In some embodiments, the TED ranges between about 0.15 mg/kg and about 0.2 mg/kg. In some embodiments, the TED ranges between about 0.2 mg/kg and about 0.225 mg/kg. In some embodiments, the TED is about 0.015, 0.02, 0.025, 0.03, 0.035, 0.04, 0.045, 0.05, 0.055, 0.06, 0.065, 0.07, 0.075, 0.1, 0.15, 0.2, or 0.225 mg/kg, or within a range defined by any two of the aforementioned values.

In some embodiments, the TED ranges from about 0.5 mg/m² of body surface area (BSA) to about 7.5 mg/m². In some embodiments, the TED ranges between about 0.5 mg/m² and about 1.25 mg/m². In some embodiments, the TED ranges between about 1.25 mg/m² and about 1.5 mg/m². In some embodiments, the TED ranges between about 1.5 mg/m2 and about 2 mg/m². In some embodiments, the TED ranges between about 2 mg/m² and about 2.5 mg/m². In some embodiments, the TED ranges between about 2.5 mg/m² and about 3 mg/m². In some embodiments, the TED ranges between about 3 mg/m² and about 3.5 mg/m². In some embodiments, the TED ranges between about 3 mg/m² and about 4 mg/m². In some embodiments, the TED ranges between about 4 mg/m² and about 5 mg/m². In some embodiments, the TED ranges between about 5 mg/m² and about 7.5 mg/m². In some embodiments, the TED is about 0.5, 0.75, 1, 1.25, 1.5, 2, 2.5, 3, 4, 5, 6, 7, or 7.5 mg/m², or within a range defined by any two of the aforementioned values.

Therapeutic effect is defined as a reduction in elevated levels of liver enzymes (including, for example, aspartate transaminase (AST) and alanine transaminase (ALT)) that are indicators of liver function to normal levels. In some embodiments, the reduction in elevated levels of liver enzymes is about 75-fold to about 100-fold. In some embodiments, the reduction in elevated levels of liver enzymes is about 10-fold to about 25-fold. In some embodiments, the reduction in elevated levels of liver enzymes is about 25-fold to about 75-fold. In some embodiments, the reduction in elevated levels of liver enzymes is about 50-fold to about 150-fold. In some embodiments, the reduction in elevated levels of liver enzymes is about 75-fold to about 200-fold. In some embodiments, the reduction in elevated levels of liver enzymes is about 10, 25, 50, 75, 100, 125, 150, 175, or 200-fold, or within a range defined by any two of the aforementioned values.

Therapeutic effect may include a reduction in elevated levels of aspartate transaminase (AST). In some embodiments, aspartate transaminase (AST) levels are reduced by about 5000 U/L, 4500 U/L, 4000 U/L, 3500 U/L, 3000 U/L, 2500 U/L, 2000 U/L, 1500 U/L, 1000 U/L, 750 U/L, 500 U/L, 250 U/L, 100 U/L, 90 U/L, 80 U/L, 70 U/L, 60 U/L, 50 U/L, 40 U/L, 30 U/L, 10 U/L, 9 U/L, 8 U/L, 7 U/L, 6 U/L, 5 U/L, 4 U/L, 3 U/L, 2 U/L, or 1 U/L, or by a range that is defined by any two of the aforementioned values. For example, in some embodiments, aspartate transaminase (AST) levels are reduced by between about 1 U/L and about 5000 U/L, about 1 U/L and about 2500 U/L, about 1 U/L and about 1000 U/L, about 1 U/L and about 500 U/L, about 500 U/L and about 5000 U/L, about 500 U/L and about 2500 U/L, about 500 U/L and about 1000 U/L, about 1000 U/L and about 5000 U/L, about 1000 U/L and about 2500 U/L, or about 2500 U/L and about 5000 U/L. In some embodiments, aspartate transaminase (AST) levels are reduced to normal levels. In some embodiments, aspartate transaminase (AST) levels are reduced to between about 14 U/L and about 36 U/L.

Therapeutic effect may include a reduction in elevated levels of alanine transaminase (ALT). In some embodiments, alanine transaminase (ALT) levels are reduced by about 3000 U/L, 2500 U/L, 2000 U/L, 1500 U/L, 1000 U/L, 750 U/L, 500 U/L, 250 U/L, 100 U/L, 90 U/L, 80 U/L, 70 U/L, 60 U/L, 50 U/L, 40 U/L, 30 U/L, 10 U/L, 9 U/L, 8 U/L, 7 U/L, 6 U/L, 5 U/L, 4 U/L, 3 U/L, 2 U/L, or 1 U/L, or by a range that is defined by any two of the aforementioned values. For example, in some embodiments, alanine transaminase (ALT) levels are reduced by between about 1 U/L and about 3000 U/L, about 1 U/L and about 2500 U/L, about 1 U/L and about 1000 U/L, about 1 U/L and about 500 U/L, about 500 U/L and about 3000 U/L, about 500 U/L and about 2500 U/L, about 500 U/L and about 1000 U/L, about 1000 U/L and about 3000 U/L, about 1000 U/L and about 2500 U/L, or about 2500 U/L and about 3000 U/L. In some embodiments, alanine transaminase (ALT) levels are reduced to normal levels. In some embodiments, alanine transaminase (ALT) levels are reduced to less than about 35 U/L.

In some embodiments, the therapeutic effect occurs in less than about one month. In some embodiments, the therapeutic effect occurs in about five months. In some embodiments, the therapeutic effect occurs in about one month to about two months. In some embodiments, the therapeutic effect occurs in about two months to about four months. In some embodiments, the therapeutic effect occurs in about four months to about six months. In some embodiments, the therapeutic effect occurs in less than about one month, in one month, in two months, in three months, in four months, in five months, or in six months, or within a range defined by any two of the aforementioned values.

In some embodiments, the therapeutic effect is maintained for over about 1, 2, 3, 4, 5, 6, 7, 8, 9, 10, 11, 12, 13, 14, 15, 16, 17, 18, 19, 20, 21, 22, 23, or 24 months, or 1, 2, 3, 4, 5, 6, 7, 8, 9, 10 or more years, or for a period of time that is within a range defined by any two of the aforementioned values. For example, in some embodiments, the therapeutic effect is maintained for between about 1-24 months, 1-18, 1-12, 1-6, 1-3, 1-2, 2-24, 2-18, 2-12, 2-6, 2-3, 3-24, 3-18, 3-12, 3-6, 6-24, 6-18, 6-12, 12-24, 12-18, or 18-24 months.

In some embodiments, the frequency of administration is once daily. In some embodiments, the frequency of administration is once a week. In some embodiments, the frequency of administration is once every two weeks. In some embodiments, the frequency of administration is once every three weeks. In some embodiments, the frequency of administration can be dose is once every 3 months. In some embodiments, the frequency of administration can be adjusted as desired by one of ordinary skill in the art based on parameters such as the dose, the route of administration, the disease, and the like.

In some embodiments, the GLP-1-GIP receptor agonists are administered intravenously. The concentration of the GLP-1-GIP receptor agonists in a solution for intravenous administration is about 0.0002 mg/ml to about 0.001 mg/ml. In some embodiments, the concentration is about 0.0002 mg/ml to about 0.0005 mg/ml. In some embodiments, the concentration is about 0.0005 mg/ml to about 0.001 mg/ml. In some embodiments, the concentration is about 0.0002, 0.00025, 0.0005, 0.00075, or 0.001, or within a range defined by any two of the aforementioned values.

In some embodiments, the GLP-1-GIP receptor agonists is administered orally. The dose of the GLP-1-GIP receptor agonists in a pharmaceutical composition for oral administration ranges from about 1 mg/day to about 5 mg/day. In some embodiments, the dose ranges between about 1 mg/day and about 2.5 mg/day. In some embodiments, the dose ranges between about 2.5 mg/day and about 5 mg/day. In some embodiments, the dose is about 1, 1.5, 2, 2.5, 3, 3.5, 4, 4.5, or 5, mg/day, or within a range defined by any two of the aforementioned values. In some embodiments, the composition for oral administration can be prepared into a solution for intravenous administration.

In some embodiments, the methods, pharmaceutical compositions, and kits comprise one or more GLP-1-GIP receptor agonists. In some embodiments, the methods, pharmaceutical compositions, and kits comprise two or more GLP-1-GIP receptor agonists. In some embodiments, the methods, pharmaceutical compositions, and kits comprise three or more GLP-1-GIP receptor agonists. In some embodiments, the methods, pharmaceutical compositions, and kits comprise four or more GLP-1-GIP receptor agonists. In some embodiments, the methods, pharmaceutical compositions, and kits comprise five GLP-1-GIP receptor agonists.

In one embodiment, the methods, pharmaceutical compositions, and kits relate to a combination therapy and comprise one or more additional drugs. In some embodiments of the methods, pharmaceutical compositions, and kits, the GLP-1-GIP receptor agonists are co-administered with methocarbamol, a centrally acting muscle relaxant, to reduce spasmodic biliary pain and enhance neuromuscular relaxation in patients with SOD. This therapy may further reduce reliance on narcotic analgesics and accelerate recovery in biliary dysmotility conditions. The dose of methocarbamol can be determined by one of ordinary skill in the art based on the circumstances. For example, when administered orally, the dosage is three tablets of 500 milligrams (mg) or two tablets of 750 mg (total dose of 1500 mg) four times a day. In some embodiments of the methods, pharmaceutical compositions, and kits, one or more other analgesics and/or muscle relaxants can be co-administered with the GLP-1-GIP receptor agonists.

In some embodiments, the two or more, three or more, four or more, or five GLP-1-GIP receptor agonists are administered in a sequential manner. In some embodiments, each GLP-1-GIP receptor agonist is administered at a different time, or at least two GLP-1-GIP receptor agonists are administered concurrently, or in a substantially simultaneous manner. Simultaneous administration can be accomplished, for example, by administering to the subject a single dosage form, for example a solution, pill or capsule, having a fixed ratio of each GLP-1-GIP receptor agonist or in multiple, single dosage forms for each GLP-1-GIP receptor agonist. Sequential or substantially simultaneous administration of each therapeutic agent can be effected by any appropriate route including, but not limited to, oral routes, intravenous routes, intramuscular routes, and direct absorption through mucous membrane tissues.

The route of administration of the GLP-1-GIP receptor agonists can be determined by one of ordinary skill in the art based on the circumstances. Several non-limiting routes of administrations are possible including parenteral, subcutaneous, intraarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

Additionally, in some embodiments, the pharmaceutical composition can be delivered via non-traditional routes such as sublingual films or intranasal sprays for patients with gastrointestinal intolerance or impaired oral absorption. These formulations may include permeation enhancers, mucoadhesive polymers, or rapid-dissolving excipients to facilitate transmucosal delivery.

In some embodiments, a pharmaceutical composition can comprise a combination of one or more GLP-1-GIP receptor agonists. In some embodiments, the one or more GLP-1-GIP receptor agonists can be present in any dosage form. For example, in a composition comprising two GLP-1-GIP receptor agonists, both GLP-1-GIP receptor agonists can be in the same dosage form (e.g., for intravenous administration or for oral administration), or one GLP-1-GIP receptor agonist can be of one dosage form (e.g., for intravenous administration or for oral administration) and other GLP-1-GIP receptor agonists can be in a different dosage form (e.g., for intravenous administration or for oral administration).

The GLP-1-GIP receptor agonists activate GLP-1 and GIP receptors, providing a mechanism of action, and thus yielding unexpectedly and significantly superior synergistic effects for treating PCS, such as SOD and other biliary disorders, as compared to either a GLP-1 receptor agonist or a GIP receptor agonist individually. In some embodiments, when a pharmaceutical composition comprises a combination of one or more GLP-1-GIP receptor agonists, the unexpectedly and significantly superior synergistic effects are further enhanced.

The synergistic effect can range from about 1.5-fold to about 50-fold. In some embodiments, the synergistic effect is about 25-fold to about 100-fold. In some embodiments, the synergistic effect is about 50-fold to about 500-fold. In some embodiments, the synergistic effect is about 100-fold to about 1000-fold. In some embodiments, the synergistic effect is about 1.5, 2, 3, 4, 5, 6, 7, 8, 9, 10, 20, 30, 40, 50, 60, 70, 80, 90, 100, 200, 300, 400, 500, 750, or 1000-fold, or within a range defined by any two of the aforementioned values. In some embodiments, a synergistic effect allows for a reduction in the requirement of a GLP-1-GIP receptor agonists to about 25% to about 75% of the recommended dose. In some embodiments, a synergistic effect allows for a reduction in the requirement of a GLP-1-GIP receptor agonist to about 5, 10, 20, 30, 40, 50, 60, 70, 80, 90, or 95% of the recommended dose, or a value within a range defined by any two of the aforementioned values.

The pharmaceutical compositions comprise active ingredients, inactive ingredients, excipients, additives, and/or pharmaceutically acceptable carriers. Examples of additives include natural polymer compounds, inorganic salts, binders, lubricants, disintegrants, surfactants, thickeners, coating agents, pH adjusters, antioxidants, flavoring agents, preservatives, and colorants among others. Examples of other pharmaceutically acceptable carriers include liquid carriers such as water, alcohol, emulsion, and solid carriers such as a gel or a powder.

Pharmaceutical compositions for intravenous administration comprise, consist of, or consist essentially of, excipient and pharmaceutically acceptable carries including one or more of sodium chloride, dextrose, and sterile water. Pharmaceutical compositions can comprise, consist of, or consist essentially of, aqueous isotonic sterile injection solutions, which can comprise, consist of, or consist essentially of, one or more of antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Pharmaceutical compositions for oral administration can be any dosage form that is suitable for oral ingestion, for example, liquid compositions such as elixir, suspension, syrup, emulsion, ampoule, etc., solid compositions such as gel, gum, drop, powder, granule, pill, sugar-coated tablet, film-coated tablet, capsule, package agent, etc. Also contemplated are sustained-release compositions such as gel-coated compositions, multi-coated compositions, localized release compositions.

In some embodiments, the pharmaceutical compositions are administered by intravenous infusion. The pharmaceutical compositions can be presented in unit-dose or multi-dose sealed containers, such as ampules and/or vials. Injection solutions and suspensions can be prepared from sterile powders, granules, and/or tablets.

In some embodiments, the pharmaceutical composition is formulated as one or more of a nanoparticle formulation, a liposomal formulation, or folic acid receptor conjugates. Nanoparticle formulations have many advantages over traditional dosage forms, such as enhanced dissolution properties and potential for efficient intracellular delivery of drugs. Nanoparticles have unique physical and chemical properties that offer several advantages as drug delivery carriers, or ‘nano-carriers.’ Nanoparticles-based composition for detection and/or treatment of cancers can comprise, consist of, or consist essentially of, nanoparticles in the form of, without limitations, quantum dots, magnetic nanoparticles, gold nanoshells (which are useful in detecting tumors and metastasis in many solid tumors), poly (lactide-co-glycolide) (PLGA)-based nanoparticles (e.g., PLGA/montmorillonite (PLGA/MMT) nanoparticles, Vitamin E-TPGS-emulsified PLGA nanoparticles, PLGA-mPEG nanoparticles), dendrimers, and SPIO and USPIO nanoparticles. Pharmaceutical compositions can also be formulated as nano-micelles-based compositions. Liposomal formulations comprise liposomes that are used as vehicles for administration of drugs. Liposomes are most often composed of phospholipids, especially phosphatidylcholine, but may also include any lipids that are compatible with a lipid bilayer structure (e.g., as egg phosphatidylethanolamine). A liposomal formulation can comprise liposomes that may employ surface ligands for attaching to unhealthy tissue. The major types of liposomes are multilamellar vesicle with several lamellar phase lipid bilayers, small unilamellar liposome vesicle with one lipid bilayer, large unilamellar vesicle, and cochleate vesicle.

In some embodiments, the nanoparticles can be one or more nanospheres, nanocylinders, nanoplates, nanoshells, nanorods, nanorices, nanofibers, nanowires, nanopyramids, nanoprisms, nanostars, nanocrescents, nanorings, and nanoantennas. In some embodiments, the dimensions of the nanoparticles can range from about 1 nm to about 100 nm. In some embodiments, the dimensions of the nanoparticles can range from about 100 nm to about 250 nm. In some embodiments, the dimensions of the nanoparticles can range from about 20 nm to about 1000 nm. In some embodiments, the dimensions of the nanoparticles can range from about 4 nm to about 6250 nm. In some embodiments, the dimensions of the nanoparticles is about 1, 5, 10, 20, 30, 40, 50, 60, 70, 80, 90, 100, 200, 300, 400, 500, 750, 1000, 1250, 2500, 3750, 5000, 7500, 10000 nm, or a value within a range defined by any two of the aforementioned values. In some embodiments, the amount of modulator and/or the amount of anti-cancer agent that can be incorporated within a nanoparticle depends on the size of the nanoparticle. Thus, the greater the size of a nanoparticle, the greater the amount of one or more modulators and/or anti-cancer agent that can be incorporated within the nanoparticle.

In some embodiments, the nanoparticles comprise one or more stabilizers, which comprise the structural components of the nanoparticle. Non-limiting examples of stabilizers include deoxycholic acid, polyvinyl alcohol (PVA), Polyvinylpyrrolidone (PVP), and polyethylene glycol (PEG). Stabilizers and their use in the production of nanoparticles are well known to one of ordinary skill in the art.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. To “treat” a disease as the term is used herein, means to reduce the frequency or severity of at least one sign or symptom of a disease or disorder experienced by a subject.

As used herein, the term “composition” has its usual meaning and thus can refer to a chemical or biological compound or substance, or a mixture or combination of two or more such compounds or substances.

The term “administering” includes contact with the skin, tissue, mucus, or fluid of a subject. It includes ingestion, inhalation, topical contact, administration as a suppository, intravaginal, intravenous, intraperitoneal, intramuscular, intralesional, intratumoral, intrathecal, intranasal, or subcutaneous administration, or the implantation of a slow-release device, such as a mini-osmotic pump, to a subject. Administration can be by any route unless specifically stated, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intra-tumoral, intraventricular, and intracranial. Other modes of delivery include, but are not limited to, the use of liposomal formulations, intravenous infusion, and transdermal patches. By “co-administer,” it is meant that a first compound described herein is administered at the same time, just prior to, or just after the administration of a second compound described herein. The preferred administration of the present disclosure comprises contact with a subject's reproductive glands or tissues, such as through transvaginal delivery and/or intravaginal delivery.

As used herein, “excipient” may include a pharmacologically inactive substance or compound that is formulated in combination with a pharmacologically active ingredient of a pharmaceutical composition, wherein an excipient may include a bulking agent, a filler, a diluent, or a product for drug solubility, drug absorption, or for modulating a pharmacokinetic property of an active ingredient of a pharmaceutical composition.

As used herein, “therapeutically effective amount” may include an amount or quantity of a pharmaceutical composition that elicits a desired clinical or biological response in a cell, a tissue, an organ, an animal or a human, or an amount or quantity that is clinically useful for reducing, eliminating, or otherwise affecting a disease, condition, or medical complication in a subject.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

### Examples

The following example is given for the purpose of illustrating various embodiments of the disclosure and are not meant to limit the present disclosure in any fashion. One skilled in the art will readily appreciate that the present disclosure is well adapted to carry out the objects and obtain the ends and advantages mentioned and those objects, ends, and advantages inherent herein. Changes therein and other uses which are encompassed within the spirit of the disclosure, as defined by the scope of the claims, will occur to those skilled in the art. Examples of some embodiments containing specific biomaterials, manufacturing methods for these embodiments, and testing these embodiments for desired physical properties and device efficacy are illustrative and not intended to limit the present disclosure in any fashion.

### Example 1—Case Study

Clinical data are presented of a 51-year-old female patient with a history of post-cholecystectomy biliary dysfunction, enlarged liver due to SOD, and recurrent hospitalizations due to right upper quadrant pain and abnormal liver enzymes. The patient's bile ducts were obliterated by a predominantly neutrophilic with some lymphocytic infiltrates and fibrinous necrosis consistent with obliterative cholangitis confirmed on liver biopsy in May 2021. The patient was also on two to three OxyContin® a day along with Dilaudid® to manage Right Upper Quadrant (RUQ, referring to discomfort or pain in the upper right quadrant of the abdomen, below the ribs and above the waistline).

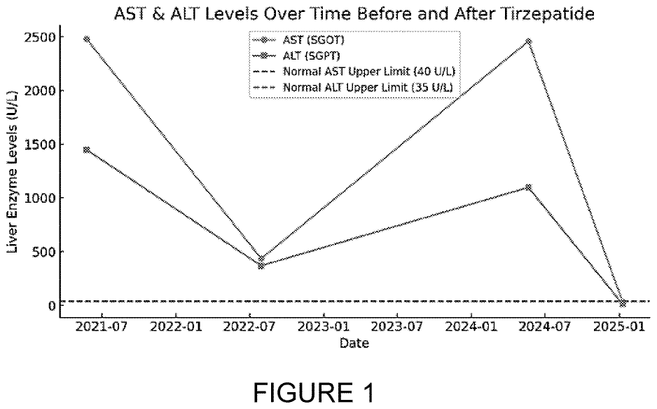

Prior to GLP-1-GIP therapy, the patient exhibited severely elevated ALT and AST levels due to SOD-related biliary dysfunction as shown by the data in FIG. 1 and Table 2.

TABLE 2

| | | |
|---|---|---|
| Date | AST (U/L) | ALT (U/L) |

Before Tirzepatide Administration

| | | |
|---|---|---|
| May 2021 | 2,479 | 1,447 |
| July 2022 | 434 | 368 |
| May 2024 | 2,458 | 1,097 |

After Tirzepatide Administration (beginning August 2024)

| | | |
|---|---|---|
| March 2025 | 16 | 11 |

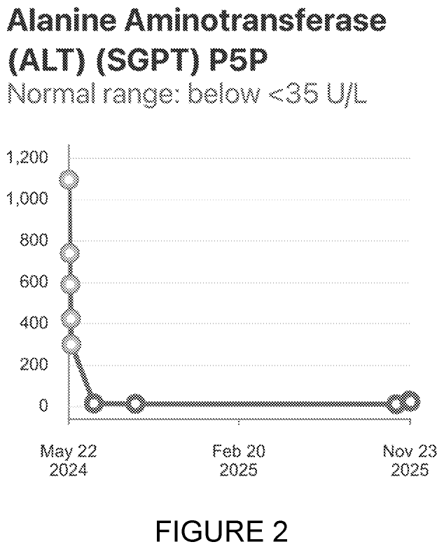

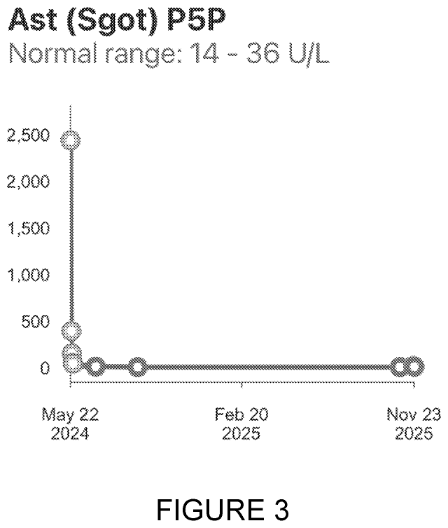

Tirzepatide was administered to the patient at a dose of 2.5 mg each day starting on day 1. Within one month of administration of tirzepatide, the patient's liver enzymes normalized. Remarkably, after fifteen months of daily administration, patient continued to have remarkable results. As shown by the data in FIGS. 1-3, which are graphical representations of AST (SGOT) and ALT (SGPT) levels over time, the patient showed unexpected and surprising improvement in AST and ALT levels within seven months of starting tirzepatide treatment. The patient's AST reduced from 2,458 U/L to 32 U/L (Normal: 14-36 U/L) and ALT reduced from 1,097 U/L to 10 U/L (Normal: <35 U/L). These values show continued normalization and stabilization of liver enzymes seven months into tirzepatide therapy. There was no further need for ERCP, narcotics, or surgical intervention. There was no recurrence of symptoms over fifteen months, representing the lowest recorded enzyme levels in four years of monitoring and highlight absence of narcotic or surgical needs. The patient reported complete resolution of spasmodic RUQ pain, normalization of liver enzymes, cessation of opioid dependence, and overall improvement in energy, sleep, and mental clarity. These results suggest a unique neuromodulatory benefit in addition to local sphincter and hepatic effects. Additionally, the administration of tirzepatide further resulted in significant liver repair as demonstrated by a liver elastography (fibroscan), which showed very low likelihood of significant fibrosis.

The data demonstrate a remarkable response to GLP-1-GIP therapy. The data show that GLP-1-GIP receptor agonists provide an unexpected and surprising non-surgical, long-term therapeutic approach for SOD and other biliary disorders, demonstrating for the first time that GLP-1-GIP agonists can effectively prevent biliary dysfunction, normalize liver enzyme levels, and reduce liver enlargement due to chronic biliary congestion. This demonstrates long-term biochemical normalization and supports the sustained efficacy of GLP-1-GIP therapy in managing both obstructive and inflammatory components of SOD and biliary disease. Notably, no further ERCP, stent, surgical intervention, or narcotic therapy have been required since initiation of tirzepatide.

Any titles or subheadings used herein are for organizational purposes and should not be used to limit the scope of embodiments disclosed herein. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

The scope of the present disclosure is not intended to be limited by the specific disclosures of preferred examples in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive. Although certain dependent claims may be described as depending from a single preceding claim, it is to be understood that each such dependent claim may alternatively be construed as depending from any one of the preceding claims, whether independent or dependent, unless expressly stated otherwise. All such alternative dependencies and combinations are expressly contemplated as part of the disclosure.

Although embodiments described herein have been disclosed in the context of certain embodiments and examples, those skilled in the art will understand that the present disclosure extends beyond the specifically disclosed embodiments to other alternative embodiments and/or uses of the disclosure and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments. Thus, it is intended that the scope of the present disclosure should not be limited by the particular disclosed embodiments described above.

It should be understood, however, that this detailed description, while indicating preferred embodiments, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods, and related components. Furthermore, embodiments may comprise several novel features, no single one of which is solely responsible for its desirable attributes or is believed to be essential to practicing the embodiments herein described.

1. A method for preventing, ameliorating, or treating post-cholecystectomy syndrome (PCS), the method comprising:
identifying a patient in need thereof; and
administering to the patient a therapeutically effective dose of a GLP-1-GIP receptor agonist,
thereby preventing, ameliorating, or treating at least one symptom or underlying condition associated with PCS.

2. The method of claim 1, wherein the at least one symptom or underlying condition associated with PCS is one or more biliary or non-biliary complications associated with PCS.

3. The method of claim 2, wherein the one or more biliary complications comprise bile duct injury, bile duct stricture, Sphincter of Oddi dysfunction, bile acid malabsorption, cystic duct remnant syndrome, or retained common bile duct stones.

4. The method of claim 1, comprising reducing sphincter spasms, biliary obstruction, and hepatobiliary congestion.

5. The method of claim 1, comprising managing post-cholecystectomy biliary dysfunction and bile duct motility disorders.

6. The method of claim 1, wherein the GLP-1-GIP receptor agonist exerts an effect through vagus nerve modulation, anti-inflammatory mechanisms, smooth muscle relaxation, improved bile flow and metabolism, and/or normalization of liver enzymes.

7. The method of claim 1, comprising maintaining long-term stability of liver enzymes, liver volume reduction, and liver fibrosis reduction.

8. The method of claim 1, wherein administration of the GLP-1-GIP receptor agonist results in normalization of ALT/AST levels, resolution of abdominal pain, avoidance of ERCP or surgery, reduction in opioid use.

9. The method of claim 1, wherein the therapeutically effective dose ranges from about 1 mg/day to about 15 mg/day.

10. The method of claim 1, wherein the GLP-1-GIP receptor agonist is tirzepatide.

11. The method of claim 1, further comprising co-administering methocarbamol to enhance neuromuscular relaxation and reduce spasmodic biliary pain.

12. The method of claim 1, further comprising administering methocarbamol to treat smooth muscle-related or spasmodic abdominal pain.

13. A pharmaceutical composition for preventing, ameliorating, or treating a post-cholecystectomy syndrome (PCS), the pharmaceutical composition comprising:
a dose of a GLP-1-GIP receptor agonist; and
a pharmaceutically acceptable carrier.

14. The pharmaceutical composition of claim 13, wherein the at least one symptom or underlying condition associated with PCS is one or more biliary or non-biliary complications associated with PCS.

15. The pharmaceutical composition of claim 14, wherein the one or more biliary complications comprise bile duct injury, bile duct stricture, Sphincter of Oddi dysfunction, bile acid malabsorption, cystic duct remnant syndrome, or retained common bile duct stones.

16. The pharmaceutical composition of claim 13, wherein the dose ranges from about 1 mg/day to about 15 mg/day.

17. The pharmaceutical composition of claim 13, wherein the dose is 2.5 mg/day.

18. The pharmaceutical composition of claim 13, wherein the GLP-1-GIP receptor agonist is tirzepatide.

19. The pharmaceutical composition of claim 13, formulated as an oral solution, sublingual film, intranasal spray, or intravenous formulation.

20. The pharmaceutical composition of claim 13, further comprising methocarbamol formulated for co-administration with the GLP-1-GIP receptor agonist.
